(19) United States
(12) Patent Application Publication
(10) Pub. No.: US 2015/0096559 A1
(43) Pub. Date: Apr. 9, 2015

(54) MANUAL VENTILATION FEEDBACK SENSOR FOR USE IN CLINICAL AND TRAINING SETTINGS

(71) Applicant: THE JOHNS HOPKINS UNIVERSITY, Baltimore, MD (US)

(72) Inventors: JORDAN DUVAL-ARNOULD, BALTIMORE, MD (US); JULIE CAMPBELL, BALTIMORE, MD (US); ELIZABETH HUNT, BALTIMORE, MD (US)

(73) Assignee: THE JOHNS HOPKINS UNIVERSITY, BALTIMORE, MD (US)

(21) Appl. No.: 14/507,009

(22) Filed: Oct. 6, 2014

Related U.S. Application Data
(60) Provisional application No. 61/887,162, filed on Oct. 4, 2013.

(51) Int. Cl. A61M 16/00; G09B 23/28; A61M 16/20

(52) U.S. Cl. CPC A61M 16/0078; A61M 16/208; G09B 23/288; A61M 2016/0027; A61M 2016/0033; A61M 2205/583
USPC 128/202.22; 434/265

(57) ABSTRACT

A manual ventilation feedback sensor for use in clinical and training settings is disclosed. Namely, a manual resuscitator device is disclosed that comprises a bag valve mask, a one-way valve, a manual ventilation bag, and a sensing module, wherein the sensing module can comprise a pressure sensor and/or flow transducer. Sensing module may further comprise a controller for processing information from the pressure sensor and/or flow transducer; namely for determining and indicating a ventilation rate. Indicators are provided to guide the user with respect to a target or desired ventilation rate.

### CROSS-REFERENCE TO RELATED APPLICATIONS

This application claims the benefit of U.S. Provisional Application No. 61/887,162, filed Oct. 4, 2013, entitled “Manual Ventilation Feedback Sensor for Use in Clinical and Training Settings,” which is incorporated herein by reference in its entirety.

### TECHNICAL FIELD

The presently disclosed subject matter relates generally to manual resuscitator devices and more particularly to a manual ventilation feedback sensor for use in clinical and training settings.

### BACKGROUND

At a staggering rate of nearly 450,000 deaths per year, more American deaths can be attributed to cardiac arrest than breast cancer, prostate cancer, house fires, firearms, traffic accidents, and AIDS combined (Cardiac Science, accessed 2013). Although healthcare providers complete resuscitation training, outcomes remain poor. Survival rates in the U.S. remain below 15% (Berdowski et al., 2010; Nichol et al., 2008; Merchant et al., 2011), while reported global survival rates range from 2-11% (Berdowski et al., 2010).

Experimental data recently suggested that rescue breathing during resuscitation may worsen patient outcome (Aufderheide et al., 2004). This observation likely is due to interruptions in chest compressions for delivery of rescue breaths during two-person cardiopulmonary resuscitation (CPR). In multi-provider resuscitation efforts, however, during which chest compressions and ventilations are provided continuously, it has been shown that providers consistently hyperventilate cardiac arrest patients during resuscitation efforts (Aufderheide et al., 2004). Hyperventilation is associated with significantly higher mean intratracheal pressures, significantly lower coronary perfusion pressures, significantly higher right atrial diastolic pressure, and significantly reduced survival rates in animal studies (Aufderheide et al., 2004; Aufderheide and Lurie, 2004). Although few studies have researched the effects of hyperventilation on humans due to ethical reasons, the equivalent animal studies demonstrate clear detriments to neural, cardiovascular, and overall outcomes with hyperventilation. The American Heart Association (AHA) recommends continuous ventilations at a rate of 8 to 10 breaths per minute in cardiac arrest patients with a secured airway (AHA, 2010). Ventilation rates observed during clinical resuscitation varied from 21 to 30 breaths per minute (Aufderheide et al., 2004; Aufderheide and Lurie, 2004; O'Neill and Deakin, 2007).

### SUMMARY

In some aspects, the presently disclosed subject matter provides a manual resuscitator device comprising a bag valve mask, a manual ventilation bag, and a pressure sensor or flow transducer, wherein the pressure sensor or flow transducer is positioned in line between the bag valve mask and the manual ventilation bag or at a pressure port of the manual ventilation bag, and wherein the pressure sensor or flow transducer is contiguous with a passage way of air flow from the manual ventilation bag to the bag valve mask, such that the pressure sensor or flow transducer is capable of measuring a streaming pressure or air flow output value between the manual ventilation bag and the bag valve mask, wherein the streaming pressure or air flow output value is indicative of a ventilation rate of the device.

Certain aspects of the presently disclosed subject matter having been stated hereinabove, which are addressed in whole or in part by the presently disclosed subject matter, other aspects will become evident as the description proceeds when taken in connection with the accompanying Drawings as best described herein below.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 illustrates a perspective view of an example of the presently disclosed manual resuscitator device comprising a pressure and/or flow sensing mechanism;

FIG. 2 illustrates a perspective view of another configuration of the presently disclosed manual resuscitator device comprising a pressure and/or flow sensing mechanism;

FIG. 3 illustrates a schematic diagram of the presently disclosed manual resuscitator device comprising a pressure and/or flow sensing mechanism;

FIG. 4 illustrates a perspective view of an example of the pressure and/or flow sensing mechanism of the presently disclosed manual resuscitator device according to one configuration;

FIG. 5 and FIG. 6 illustrate block diagrams of yet other embodiments of the pressure and/or flow sensing mechanism of the presently disclosed manual resuscitator device; and

FIG. 7 illustrates a flow diagram of an embodiment of a method of operation of the presently disclosed manual resuscitator device comprising a pressure and/or flow sensing mechanism.

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a manual ventilation feedback sensor for use in clinical and training settings. Namely, a manual resuscitator device comprises a pressure and/or flow sensing mechanism, wherein the pressure and/or flow sensing mechanism senses pressure and/or flow changes generated by the manual deflation of a manual ventilation bag. In some embodiments, the pressure and/or flow sensing mechanism is a pressure sensor and/or flow transducer. In some embodiments, information from the pressure and/or flow sensing mechanism is used to calculate and display the ventilation rate to the healthcare provider. Providing healthcare providers with real-time ventilation rate feedback using the presently disclosed manual ventilation feedback sensor could prevent or minimize the risk of hyperventilation of patients. In some embodiments, the presently disclosed manual resuscitator device is capable of delivering a continuous ventilation at a rate of from about 8 to about 10 breaths per minute.

Accordingly, in some embodiments, the presently disclosed manual resuscitator device comprises a pressure and/or flow sensing mechanism that connects in line between a manual ventilation bag (e.g., an artificial manual breathing unit (Ambu) bag), which is used to provide positive pressure ventilation, and a bag valve mask (BVM) or intubation tube.

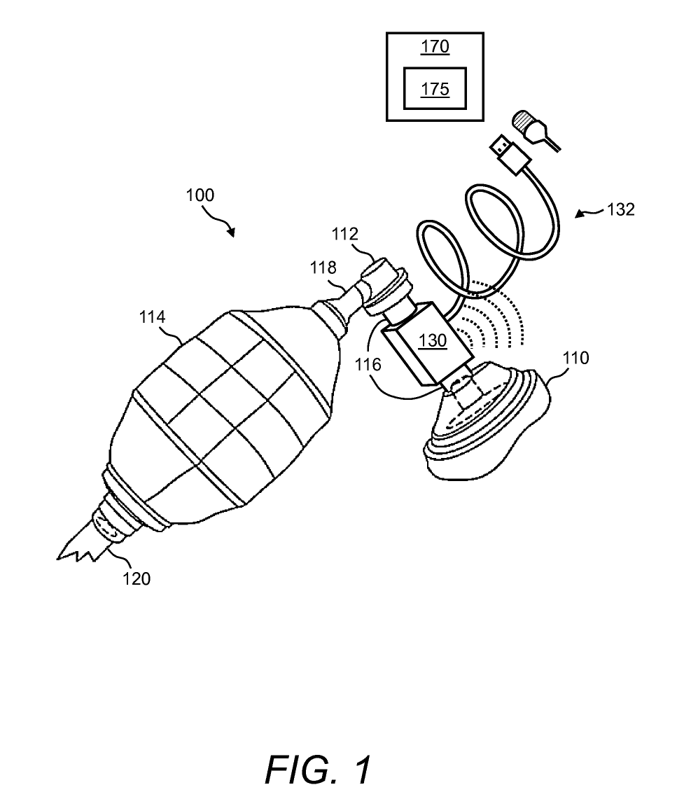

Referring now to FIG. 1 is a perspective view of an example of the presently disclosed manual resuscitator device 100 comprising a pressure and/or flow sensing mechanism. For example, manual resuscitator device 100 comprises a bag valve mask 110, a one-way valve 112, and a manual ventilation bag 114 (e.g., an Ambu bag). Namely, bag valve mask 110 and one-way valve 112 are coupled via an air flow line 116 and one-way valve 112 is coupled to one end of manual ventilation bag 114 via an air flow line 118. The end of manual ventilation bag 114 opposite air flow line 118 has an air input port 120 for receiving ambient air or an oxygen supply. In other configurations, manual resuscitator device 100 includes an intubation tube (not shown) in place of bag valve mask 110.

In manual resuscitator device 100, a sensing module 130 is provided in the flow path of air flow line 116 between bag valve mask 110 and one-way valve 112, i.e., sensing module 130 is downstream of one-way valve 112. In another configuration of manual resuscitator device 100, sensing module 130 can be provided in the flow path of air flow line 118, i.e., sensing module 130 is upstream of one-way valve 112 (not shown). In yet another configuration of manual resuscitator device 100, sensing module 130 can be provided at a pressure port (not shown) of manual ventilation bag 114.

In still another configuration of manual resuscitator device 100, sensing module 130 can be provided in line with other respiratory devices including, but not limited to, an end tidal CO₂ cuvette (e.g., an infrared CO₂ sensor). For example, when a provider performing CPR tires, the patient's end-tidal CO₂ (ETCO₂), i.e., the level of carbon dioxide released at the end of expiration, decreases and then increases when a fresh provider takes over. Further, when a patient experiences a return of spontaneous circulation, an initial indication can be a sudden increase in ETCO₂. Likewise, a sudden drop in ETCO₂ can indicate that the patient is losing pulses and CPR might need to be re-initiated. Accordingly, measurement of ETCO₂ can be used to monitor the effectiveness of CPR. In such configurations, sensing module 130 is contiguous with the passage way of air flow from manual ventilation bag 114 to bag valve mask 110.

Sensing module 130 includes a housing through which air flow line 116 passes and for holding sensing devices. For example, sensing module 130 can include any components or mechanisms for measuring air pressure or air flow in air flow line 116. Further, in some configurations, sensing module 130 operates in standalone mode. In other configurations, sensing module 130 includes a wired or wireless communications link 132 for exchanging information with any external computing device, such as computing device 170. Software 175 may be installed on computing device 170 for processing information from sensing module 130 of manual resuscitator device 100.

Computing device 170 can be any computing device that is capable of executing program instructions and communicating with sensing module 130 of manual resuscitator device 100. Computing device 170 can be, for example, a desktop computer, a laptop computer, a handheld computing device, a personal digital assistant (PDA), a tablet device, a mobile phone (e.g., a smart phone), and the like. Depending on the type of computing device 170, software 175 can be implemented as a desktop application or a mobile app. Further, computing device 170 can be a healthcare simulation training device that is used to link user performance to simulated patient characteristics.

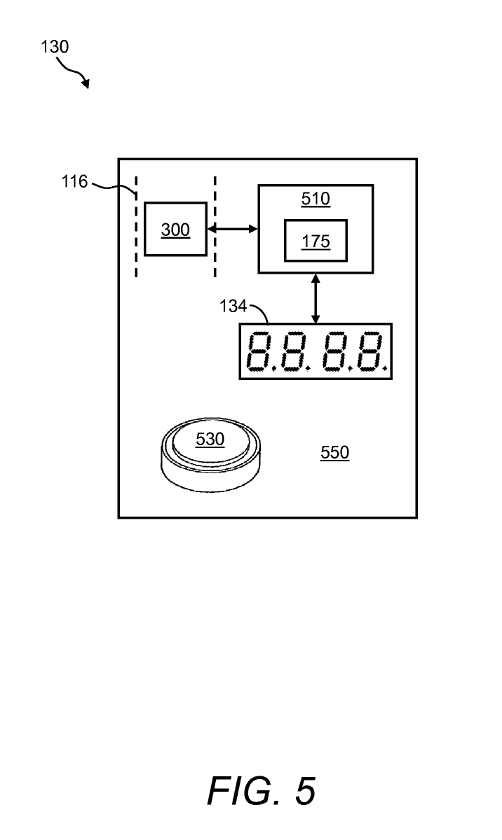

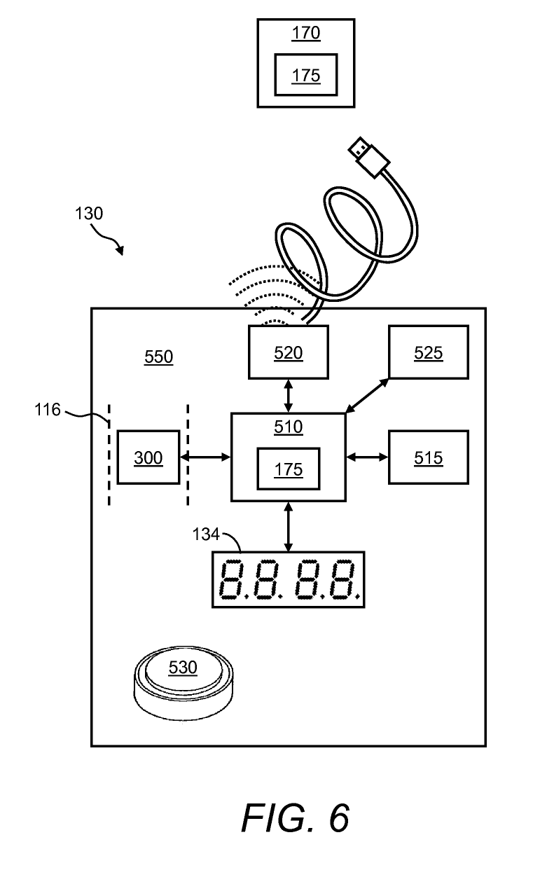

In one example, using information (i.e., feedback) from sensing module 130, software 175 can be used to calculate one or more ventilation characteristics selected from the group consisting of a rate, a pressure, a frequency, and a volume. In other configurations, software 175 resides inside sensing module 130 of manual resuscitator device 100, more details of which are shown and described with reference to FIG. 5 and FIG. 6.

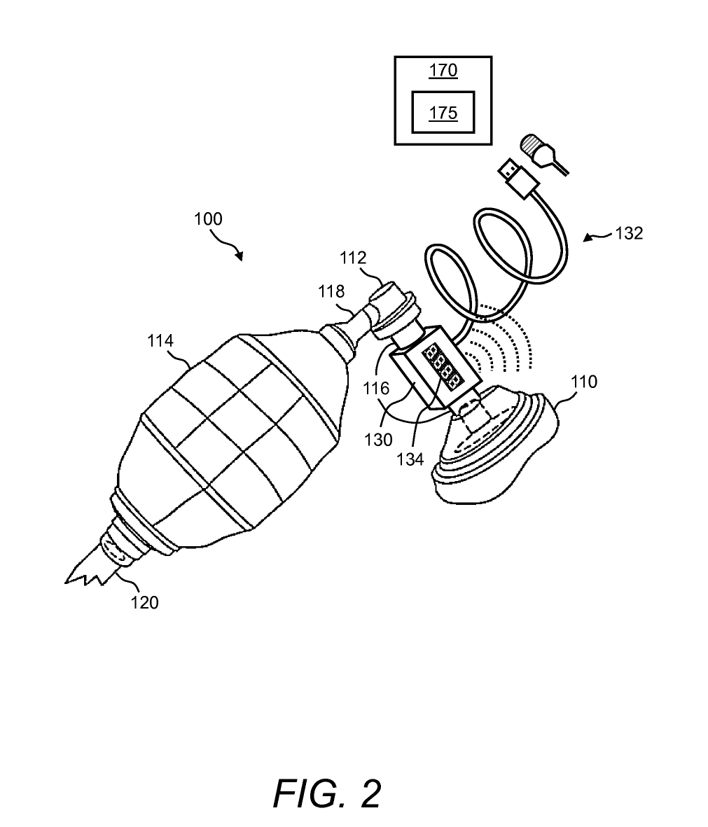

Referring now to FIG. 2, in yet another configuration of manual resuscitator device 100, sensing module 130 can include a digital display 134 for indicating the amount of air pressure or air flow detected in, for example, air flow line 116. Digital display 134 can be any type of display, such as a light-emitting diode (LED) display or a liquid crystal display (LCD). Using digital display 134, the air pressure, air flow rate, and/or the ventilation rate can be displayed to the user.

Sensing module 130 can include a pressure sensor and/or a flow transducer, such as those comprising a piezoresistive device. The sensing components of sensing module 130 generally act as a transducer and generate an electrical signal as a function of the air pressure and/or air flow imparted thereon. Representative, non-limiting pressure sensors and flow transducers include, but are not limited to, a monolithic silicon piezoresistive transducer, e.g., such as the MPxx5010 series integrated silicon pressure sensor available from Freescale Semiconductor, Inc. (Austin, Tex.), an example of which is the model MPX5010GSX sensor; a silicon piezoresistive pressure sensor, such as a Honeywell integrated pressure transducer available from Honeywell International, Inc. (Plymouth, Minn.); the Honeywell Zephyr™ analog airflow sensors; and the Honeywell Zephyr™ digital airflow sensors.

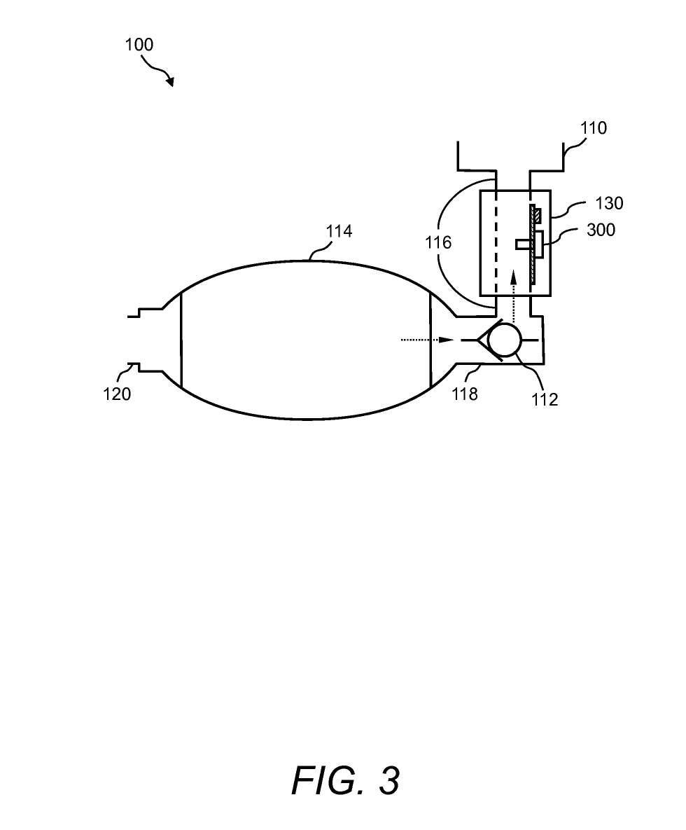

Referring now to FIG. 3 is a schematic diagram of the presently disclosed manual resuscitator device 100 comprising sensing module 130, which is the pressure and/or flow sensing mechanism. FIG. 3 shows a pressure and/or flow sensing device 300 installed in relation to an air flow path, such as air flow line 116. The housing of sensing module 130 provides a sealed compartment in which pressure and/or flow sensing device 300 is contiguous with airflow circuit, but does not interrupt the airflow.

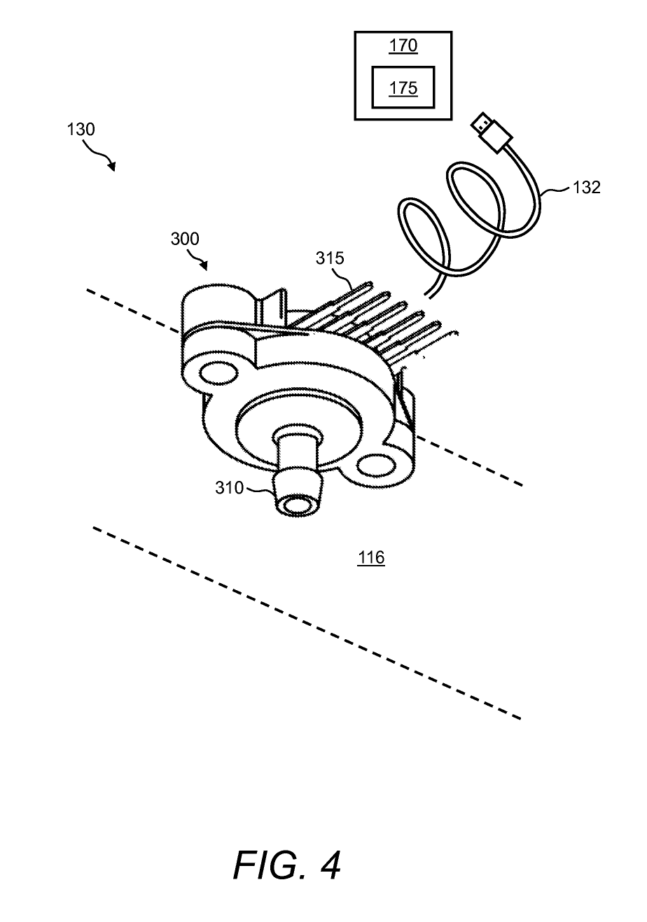

In one configuration, sensing module 130 includes a discrete pressure and/or flow sensing device 300 that can be electrically coupled to an external computing device, such as computing device 170. An example of a configuration of sensing module 130 is shown in FIG. 4. Referring now to FIG. 4, sensing module 130 includes pressure and/or flow sensing device 300 alone. Pressure and/or flow sensing device 300 can be any discrete pressure and/or flow sensing device, such as any pressure sensor or flow transducer (e.g., those comprising a piezoresistive device). A sensing port 310 of pressure and/or flow sensing device 300 is positioned in the air way of air flow line 116. Pressure and/or flow sensing device 300 acts as a transducer and generate an electrical signal as a function of the air pressure and/or air flow imparted thereon. Electrical input/output (I/O) pins 315 can be wired to, for example, computing device 170, wherein software 175 of computing device 170 can process the information received from pressure and/or flow sensing device 300. By contrast, sensing module 130 can include a partial or full complement of electronics to process the information received from pressure and/or flow sensing device 300, examples of which is shown and described herein below with reference to FIG. 5 and FIG. 6.

Referring now to FIG. 5 is a block diagram of another example of sensing module 130, which is the pressure and/or flow sensing mechanism of the presently disclosed manual resuscitator device 100. In this example, sensing module 130 includes pressure and/or flow sensing device 300, a controller 510, digital display 134, and a power source in the form of a battery 530. Pressure and/or flow sensing device 300, controller 510, digital display 134, and battery 530 are installed on a printed circuit board (PCB) 550 inside the housing of sensing module 130. Namely, using PCB 550, pressure and/or flow sensing device 300 is electrically coupled to controller 510 and controller 510 is electrically coupled to digital display 134.

Controller 510 is used to manage the overall operations of sensing module 130 with respect to, for example, calculating one or more ventilation characteristics selected from the group consisting of a rate, a pressure, a frequency, and a volume. Controller 510 can be any standard controller, processor, or microprocessor device that is capable of executing program instructions. Battery 530 can be any standard cylindrical battery, such as quadruple-A, triple-A, or double-A, or a battery from the family of button cell and coin cell batteries. In one example, battery 530 is the CR2032 coin cell 3-volt battery.

In this example, software 175 is installed on controller 510 rather than on an external computing device. Controller 510 and/or software 175 are pre-programmed to analyze the pressure or air flow output value of pressure and/or flow sensing device 300 using one or more predetermined methods. In particular embodiments, the one or more predetermined methods can be used to calculate one or more ventilation characteristics selected from the group consisting of a rate, a pressure, a frequency, and a volume. In one example, using controller 510 and software 175, the air pressure, air flow rate, and/or the ventilation rate can be displayed in real time to the user via digital display 134.

Referring now to FIG. 6 is a block diagram of yet another example of sensing module 130, which is the pressure and/or flow sensing mechanism of the presently disclosed manual resuscitator device 100. Sensing module 130 shown in FIG. 6 is substantially the same as sensing module 130 shown in FIG. 5 except that it further includes data storage 515, a communications interface 520, and one or more indicators 525.

Data storage 515 can be any volatile or nonvolatile memory device for storing any information from pressure and/or flow sensing device 300 and/or generated by software 175. In one example, each time the user squeezes manual ventilation bag 114 the pressure and/or air flow inside air flow lines 116 and 118 spikes as compared to the ambient air pressure and/or air flow. Accordingly, each high-pressure or high-flow event that is indicated by pressure and/or flow sensing device 300 is time stamped and logged in data storage 515.

Communications interface 520 may be any wired and/or wireless communication interface for connecting to a network (not shown) and by which information may be exchanged with other devices, such as computing device 170. Examples of wired communication interfaces may include, but are not limited to, USB ports, RS232 connectors, RJ45 connectors, Ethernet, and any combinations thereof. Examples of wireless communication interfaces may include, but are not limited to, an Intranet connection, Internet, ISM, Bluetooth® technology, Bluetooth® Low Energy (BLE) technology, Wi-Fi, Wi-Max, IEEE 402.11 technology, ZigBee technology, Z-Wave technology, 6LoWPAN technology (i.e., IPv6 over Low Power Wireless Area Network (6LoWPAN)), ANT or ANT+ (Advanced Network Tools) technology, radio frequency (RF), Infrared Data Association (IrDA) compatible protocols, Local Area Networks (LAN), Wide Area Networks (WAN), Shared Wireless Access Protocol (SWAP), any combinations thereof, and other types of wireless networking protocols. Examples of information facilitated by the communications interface 520 include streaming the air pressure and/or air flow value from pressure and/or flow sensing device 300 to, for example, computing device 170.

The one or more indicators 525 can be visual indicators, audible indicators, tactile indicators, and any combinations thereof. An example of visual indicators is light-emitting diodes (LEDs). An example of an audible indicator is an audio speaker. An example of a tactile indicator is a vibration mechanism.

In the configuration shown in FIG. 6, software 175 can reside at controller 510 only, can reside at computing device 170 only, or can reside at both controller 510 and computing device 170. In one example, software 175 at controller 510 and software 175 at computing device 170 can be configured in a client/server computing architecture.

Using information (i.e., feedback) from pressure and/or flow sensing device 300, software 175 at controller 510, computing device 170, or both can be used to calculate one or more ventilation characteristics selected from the group consisting of a rate, a pressure, a frequency, and a volume. In one example, using software 175, the air pressure, air flow rate, and/or the ventilation rate can be displayed in real time to the user via digital display 134.

Digital display 134 and the one or more indicators 525 can be used, for example, to guide the user with respect to a target or desired ventilation rate. For example, one or more indicators 525 can be used to indicate to the user whether, for example, the ventilation rate is too slow, too fast, or about right. At the same time, the actual ventilation rate can be displayed via digital display 134. In one example, a slowly flashing LED means the ventilation rate is too slow, a rapidly flashing LED means the ventilation rate is too fast, or a solidly lit LED means the ventilation rate is about right. In another example, a sequence of slow audible beeps means the ventilation rate is too slow, a sequence of rapid audible beeps means the ventilation rate is too fast, or no audible beeps means the ventilation rate is about right. The user may adjust his/her operation of manual resuscitator device 100 based on information displayed on digital display 134 and/or information conveyed by indicators 525.

In some embodiments, computing device 170 is a healthcare simulation training device (not shown) that is connected wirelessly to manual resuscitator device 100 and used to link user performance to simulated patient characteristics. Accordingly, the presently disclosed manual resuscitator device 100 can be used beneficially for both clinical and training purposes. For example, the presently disclosed manual resuscitator device 100 can provide access to provider ventilation performance characteristics (e.g., frequency, pressure) in training with any simulator. The presently disclosed manual resuscitator device 100 also can be used to provide clinical ventilation feedback in cases when end tidal CO₂ data are unavailable.

Further, one of ordinary skill in the art will recognize that the sensing module 130 and associated electronics/components and the like, can be used with existing bag valve masks (BVMs). Accordingly, the sensing module 130 and associated electronics/components can be used in line with an existing BVM or connected at the pressure port of an existing BVM as an add-on accessory to such BVMs and can easily be added or removed from an existing BVM without significantly modifying the BVM.

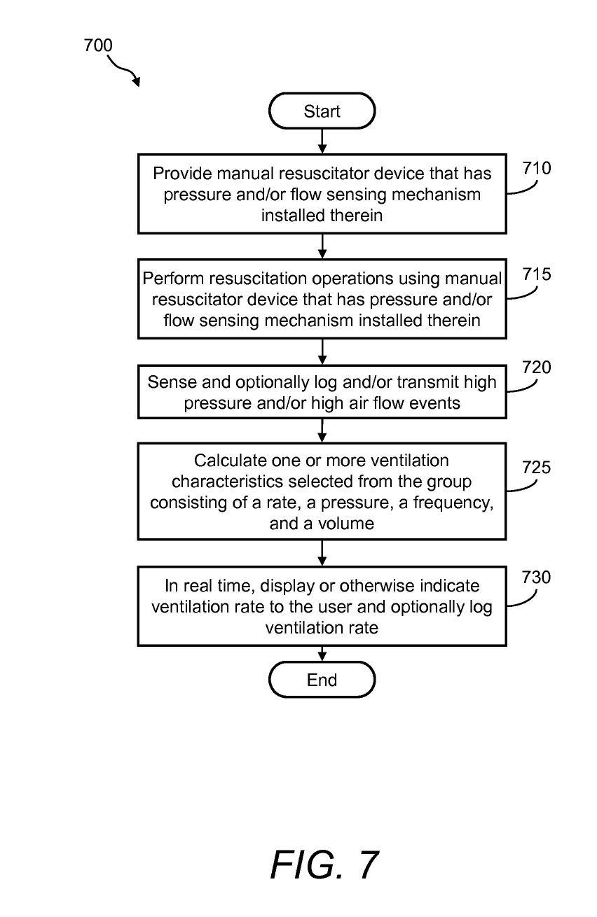

FIG. 7 illustrates a flow diagram of an example of a method 700 of operation of the presently disclosed manual resuscitator device 100 comprising sensing module 130, which is a pressure and/or flow sensing mechanism. Method 700 may include, but is not limited to, the following steps.

At a step 710, the presently disclosed manual resuscitator device 100 is provided that comprises sensing module 130, which is a pressure and/or flow sensing mechanism.

At a step 715, using the presently disclosed manual resuscitator device 100, a user performs resuscitation operations on a subject or on a subject dummy for training purposes. Namely, the user places bag valve mask 110 over the subject's mouth and nose and then squeezes manual ventilation bag 114 (e.g., the Ambu bag) at a certain ventilation rate. In one example, the AHA recommends continuous ventilations at a rate of 8 to 10 breaths per minute in cardiac arrest patients with a secured airway.

At a step 720, using pressure and/or flow sensing device 300, the high pressure and/or high air flow events are sensed and optionally logged in memory and/or transmitted to another computing device. Namely, each time the user squeezes manual ventilation bag 114, the pressure and/or air flow inside air flow lines 116 and 118 spikes as compared to the ambient air pressure and/or air flow. In one example, using software 175 at controller 510, each high-pressure and/or high-flow event that is indicated by pressure and/or flow sensing device 300 is time stamped and logged in data storage 515. In another example, using controller 510 and communications interface 520, readings from pressure and/or flow sensing device 300 are transmitted to computing device 170 and each high-pressure and/or high-flow event is time stamped and logged at computing device 170.

At a step 725, the one or more ventilation characteristics selected from the group consisting of a rate, a pressure, a frequency, and a volume are calculated. For example, based on information from pressure and/or flow sensing device 300, software 175 at controller 510 and/or software 175 at computing device 170 calculates one or more ventilation characteristics selected from the group consisting of a rate, a pressure, a frequency, and a volume. In one example, software 175 determines the amount of time between at least two high-pressure and/or high-flow events and then calculates the ventilation rate. For example, 3 seconds between events is a 20 breaths per minute rate, 5 seconds between events is a 12 breaths per minute rate, 6 seconds between events is a 10 breaths per minute rate, 10 seconds between events is a 6 breaths per minute rate, and so on.

At a step 730, in substantially real time, the ventilation rate is displayed or otherwise indicated to the user and optionally logged in data storage 515. For example, software 175 is programmed with a target or desired ventilation rate of, for example, 8 to 10 breaths per minute in cardiac arrest patients with a secured airway. Accordingly, 7.5 seconds between events correlates to 8 breaths per minute, whereas 6 seconds between events correlates to 10 breaths per minute. Therefore, the actual ventilation rate can be displayed via digital display 134, while at the same time the one or more indicators 525 can be used to indicate to the user whether, for example, the ventilation rate is too slow, too fast, or about right.

In one example, if the calculated ventilation rate is 6 breaths per minute then an LED flashes slowly to indicate that the ventilation rate is too slow. In response, the user may speed up the ventilation rate.

In another example, if the calculated ventilation rate is 20 breaths per minute then an LED flashes rapidly to indicate that the ventilation rate is too fast. In response, the user may slow down the ventilation rate.

In another example, if the calculated ventilation rate is 10 breaths per minute then an LED is lit solidly to indicate that the ventilation rate is within the desired range of 8 to 10 breaths per minute. In response, the user maintains the current ventilation rate.

Accordingly, the presently disclosed manual resuscitator device 100 can sense pressure changes generated by manual deflation of manual ventilation bag 114, e.g., Ambu bag, and calculate and display the ventilation rate to a healthcare provider. Accordingly, the presently disclosed manual resuscitator device 100 can provide feedback to prevent or minimize the risk of subject hyperventilation during resuscitation and ultimately improve cardiac arrest outcomes. In contrast to existing alternatives, the presently disclosed manual resuscitator device 100 is inexpensive, easy to implement, and healthcare providers do not have to change their current practices to incorporate the device into standard resuscitation procedures.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A manual resuscitator device comprising a bag valve mask, a manual ventilation bag, and a pressure sensor or flow transducer, wherein the pressure sensor or flow transducer is positioned in line between the bag valve mask and the manual ventilation bag or at a pressure port of the manual ventilation bag, and wherein the pressure sensor or flow transducer is contiguous with a passage way of air flow from the manual ventilation bag to the bag valve mask, such that the pressure sensor or flow transducer is capable of measuring a streaming pressure or air flow output value between the manual ventilation bag and the bag valve mask, wherein the streaming pressure or air flow output value is indicative of a ventilation rate of the device.

2. The manual resuscitator device of claim 1, further comprising an end tidal CO₂ sensor, wherein the end tidal CO₂ sensor is configured to be in line with the pressure sensor or flow transducer.

3. The manual resuscitator device of claim 1, wherein the pressure sensor or flow transducer is encased in a housing.

4. The manual resuscitator device of claim 3, wherein the housing comprises a sealed compartment in which the pressure sensor or flow transducer is contiguous with airflow circuit, but does not interrupt the airflow.

5. The manual resuscitator device of claim 1, wherein the pressure sensor or flow transducer is in electrical communication with a microprocessor configured to send a digital signal corresponding to the streaming pressure output value to a computer.

6. The manual resuscitator device of claim 1, wherein the pressure sensor or flow transducer is in electrical communication with a microprocessor unit configured to send a digital signal directly to a display unit or display monitor.

7. The manual resuscitator device of claim 6, wherein the display unit or display monitor comprises a liquid crystal display screen.

8. The manual resuscitator device of claim 1, wherein the device is in wireless communication with a healthcare simulation training device.

9. The manual resuscitator device of claim 1, wherein the microprocessor is pre-programmed to analyze the pressure or air flow output value using one or more predetermined methods.

10. The manual resuscitator device of claim 9, wherein the one or more predetermined methods can be used to calculate one or more ventilation characteristics selected from the group consisting of a rate, a pressure, a frequency, and a volume.

11. The manual resuscitator device of claim 1, wherein the device is capable of delivering a continuous ventilation at a rate of about 8 to about 10 breaths per minute.

12. The manual resuscitator device of claim 1, wherein the pressure sensor or flow transducer comprises a piezoresistive device.

13. The manual resuscitator device of claim 1, wherein the bag valve mask is replaced by an intubation tube.
